(19) United States
(12) Patent Application Publication
(10) Pub. No.: US 2026/0183574 A1
(43) Pub. Date: Jul. 2, 2026

(54) CIRCULATORY SYSTEM STIMULATION DEVICE

(71) Applicant: NATIONAL TAIWAN UNIVERSITY, Taipei City (TW)

(72) Inventors: JAW-LIN WANG, Taipei City (TW); WEN-SHIANG CHEN, Taipei City (TW)

(21) Appl. No.: 19/088,410

(22) Filed: Mar. 24, 2025

(30) Foreign Application Priority Data
Dec. 31, 2024 (TW) 113151615

(51) Int. Cl. A61N 7/00

(52) U.S. Cl. CPC A61N 7/00

(57) ABSTRACT

A circulatory system stimulation device includes a support unit, a stimulation unit disposed on the support unit for providing ultrasound stimulation, and a heart rate sensing unit electrically connected to the stimulation unit. The heart rate sensing unit detects a heart rate signal, and the ultrasound stimulation is synchronized with the heart rate signal.

### CROSS-REFERENCE TO RELATED PATENT APPLICATION

This application claims the benefit of priority to Taiwan Patent Application No. 113151615, filed on Dec. 31, 2024. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a stimulation device, and more particularly to a circulatory system stimulation device.

### BACKGROUND OF THE DISCLOSURE

With an increase in the elderly population, the number of people suffering from brain diseases such as Parkinson's disease, Alzheimer's disease, and stroke has increased in a significant manner. In particular, the stroke is ranked as the fourth leading cause of death (following cancer, heart diseases, and pneumonia) in Taiwan. Although the mortality rate of stroke patients is reduced due to medical advancements, long-term medical care still brings about a heavy burden to the society and families.

In the related art, concentrated microcurrent devices have been developed for muscle stimulation, stroke rehabilitation, and the treatment of cardiovascular diseases. These devices apply current to the head of a patient, thereby generating electrical stimulation within the brain. However, if current intensity is too high or an application duration of the current is too long, pain can be felt on the cranial surface, and causes discomfort to the patient.

Therefore, how to improve a stimulation device, so as to enhance circulation efficiency of a circulatory system stimulation device and overcome the aforementioned problems, has become one of the important issues to be solved in the relevant industry.

### SUMMARY OF THE DISCLOSURE

In response to the above-referenced technical inadequacies, the present disclosure provides a medical device that can enhance intracerebral circulation.

In order to solve the above-mentioned problems, one of the technical aspects adopted by the present disclosure is to provide a circulatory system stimulation device for stimulating a circulatory system. The circulatory system stimulation device includes: a support unit; a stimulation unit disposed on the support unit for providing ultrasound stimulation; and a heart rate sensing unit electrically connected to the stimulation unit. The heart rate sensing unit detects a heart rate signal, and the ultrasound stimulation is synchronized with the heart rate signal.

In one of the possible or preferred embodiments, the circulatory system stimulation device further includes a control unit electrically connected to the heart rate sensing unit and the stimulation unit, so as to control a stimulation frequency of the stimulation unit based on the heart rate signal detected by the heart rate sensing unit.

In one of the possible or preferred embodiments, the heart rate sensing unit detects the heart rate signal, the heart rate signal includes a systolic signal and a diastolic signal, and the ultrasound stimulation is synchronized with the systolic signal.

In one of the possible or preferred embodiments, the support unit is an annular band.

In one of the possible or preferred embodiments, the support unit includes a cap body and a cap band, the heart rate sensing unit is disposed on the cap band, and the stimulation unit is disposed on the cap body.

In one of the possible or preferred embodiments, the stimulation unit is arranged in pairs.

In one of the possible or preferred embodiments, the circulatory system is intracerebral circulation, lymphatic circulation, or blood circulation.

Therefore, in the circulatory system stimulation device provided by the present disclosure, by virtue of “the circulatory system stimulation device including the heart rate sensing unit, and the stimulation unit being disposed on the support unit and electrically connected to the heart rate sensing unit for providing the ultrasound stimulation,” and “the heart rate sensing unit detecting the heart rate signal, and the ultrasound stimulation being synchronized with the heart rate signal,” the effectiveness of stimulating the intracerebral circulation can be improved, and a patient is less likely to feel uncomfortable.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic view of a circulatory system stimulation device according to a first embodiment of the present disclosure;

FIG. 2A and FIG. 2B are each a schematic view of a circulatory system stimulation device according to a second embodiment of the present disclosure;

FIG. 3 is a schematic view of a circulatory system stimulation device according to a third embodiment of the present disclosure;

FIG. 4 is a curve graph showing intracerebral circulation of a mouse; and

FIG. 5 is a bar graph showing how intracerebral circulation efficiency is affected by a heart rate under different conditions.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

### First Embodiment

Referring to FIG. 1, a first embodiment of the present disclosure provides a circulatory system stimulation device A1 for stimulating a circulatory system. The circulatory system stimulation device A1 includes: a support unit 1, a heart rate sensing unit 2, and a stimulation unit 3. As shown in FIG. 1, the support unit 1 is an annular band, and the heart rate sensing unit 2 and the stimulation unit 3 are disposed on the support unit 1. In addition, the support unit 1 may be an elastic annular band, so as to be easily fitted onto the head of a user. In this embodiment, the heart rate sensing unit 2 and the stimulation unit 3 may be arranged adjacent to each other on the same horizontal plane. The heart rate sensing unit 2 may be used to receive heart sound signals that match a predetermined frequency range, and the received heart sound signals are processed and calculated to obtain a heart rate of the user, thereby achieving numerical quantification. For example, the heart rate sensing unit 2 is a device that is capable of sensing the heart rate, such as a smartwatch or an electrocardiogram. In the present disclosure, the circulatory system is, for example but not limited to, intracerebral circulation, lymphatic circulation, or blood circulation.

The stimulation unit 3 may be electrically connected to the heart rate sensing unit 2, and is equipped with a piezoelectric element to provide ultrasound stimulation. The circulatory system stimulation device A1 may further include a control unit (not shown in the drawings) for controlling a stimulation frequency provided by the stimulation unit 3. Specifically, the control unit may control the stimulation frequency of the stimulation unit 3 based on a heart rate signal detected by the heart rate sensing unit 2. It is worth mentioning that the ultrasound stimulation of the present disclosure is discontinuous stimulation. That is, the stimulation unit 3 may provide different frequencies and intensities based on the heart sound signals received by the heart rate sensing unit 2. In practical application, the heart rate sensing unit 2 detects the heart rate signal, and the ultrasound stimulation is synchronized with the heart rate signal.

In this embodiment, the support unit 1 may be worn on the head of the user, such that the stimulation unit 3 is attached to the temple of the user, and the heart rate sensing unit 2 stimulation unit 3 is attached to the forehead of the user. However, the aforementioned details are disclosed for exemplary purposes only, and are not meant to limit the scope of the present disclosure.

### Second Embodiment

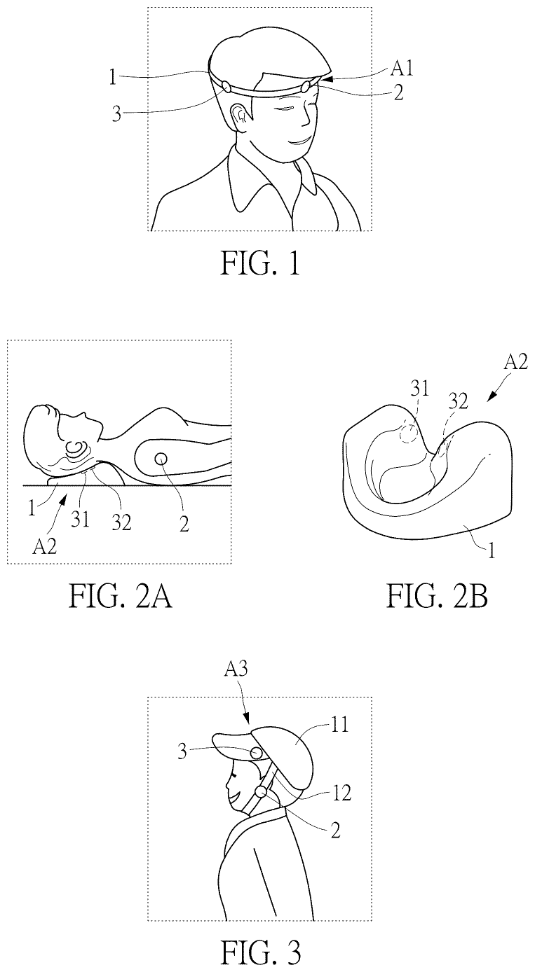

Referring to FIG. 2A and FIG. 2B, a second embodiment of the present disclosure provides a circulatory system stimulation device A2 for stimulating a circulatory system. The circulatory system stimulation device A2 includes: a support unit 1, a heart rate sensing unit 2, and a stimulation unit 3. As shown in FIG. 2A and FIG. 2B, the support unit 1 is pillow-shaped and has a groove, and the stimulation unit 3 includes a first stimulation unit 31 and a second stimulation unit 32. In other words, the stimulation unit 3 is arranged on the support unit 1 in pairs. In the present disclosure, the stimulation unit 3 is in contact with the head of the user. Preferably, the stimulation unit 3 is in contact with the scalp of the user.

In this embodiment, the heart rate sensing unit 2 may be an individual patch-like unit that is adhered to the body of the user. For example, the heart rate sensing unit 2 is adhered to the forehead, the arm, or any other location where the heart rate signal can be detected. In addition, in this embodiment, the user may continuously use the circulatory system stimulation device A2 during sleep, thereby expanding an application range of the circulatory system stimulation device A2. However, the aforementioned details are disclosed for exemplary purposes only, and are not meant to limit the scope of the present disclosure.

### Third Embodiment

Referring to FIG. 3, a third embodiment of the present disclosure provides a circulatory system stimulation device A3 for stimulating a circulatory system. The circulatory system stimulation device A3 includes:
a support unit 1, a heart rate sensing unit 2, and a stimulation unit 3. As shown in FIG. 3, the support unit 1 may include a cap body 11 and a cap band 12, the heart rate sensing unit 2 is disposed on the cap band 12, and the stimulation unit 3 is disposed on the cap body 11.

In this embodiment, the circulatory system stimulation device A3 is cap-shaped and may be worn on the head of the user, such that the stimulation unit 3 is attached to the head of the user, and the heart rate sensing unit 2 is attached to the cheek of the user. In addition, in this embodiment, multiple stimulation units 3 may be included. Furthermore, the cap body 11 may include a head circumference adjustment unit (not shown in the drawings) to adjust the elasticity of the cap body 11 according to a head circumference of the user. However, the aforementioned details are disclosed for exemplary purposes only, and are not meant to limit the scope of the present disclosure.

### Experimental Example

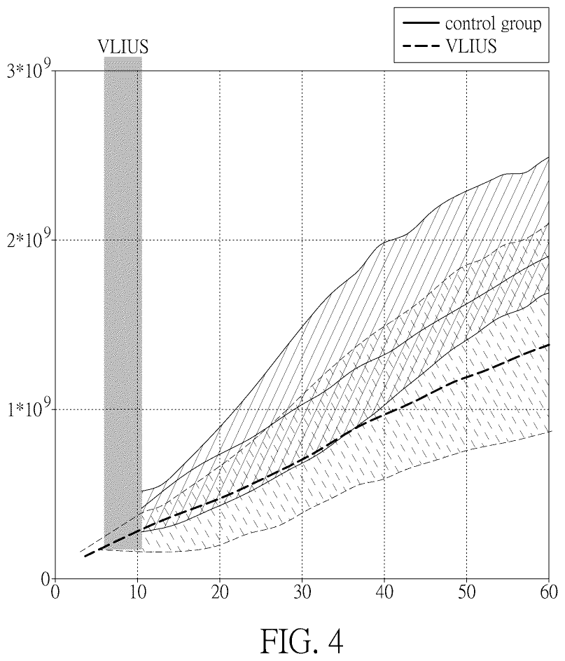

Referring to FIG. 4, FIG. 4 is a curve graph showing intracerebral circulation of a mouse, and will be used to illustrate the relationship between ultrasound stimulation and the intracerebral circulation. As shown in FIG. 4, while the ultrasound stimulation is not applied to the brain of the mouse in a control group, the ultrasound stimulation is applied to the brain of the mouse in an ultrasound stimulation group (with very low-intensity ultrasound). According to the results in FIG. 4, pixel strength of the ultrasound stimulation group is larger than that of the control group. In other words, an intracerebral circulation rate of the ultrasound stimulation group is larger than that of the control group. That is, in the present disclosure, it is proven that the ultrasound stimulation can indeed enhance the intracerebral circulation.

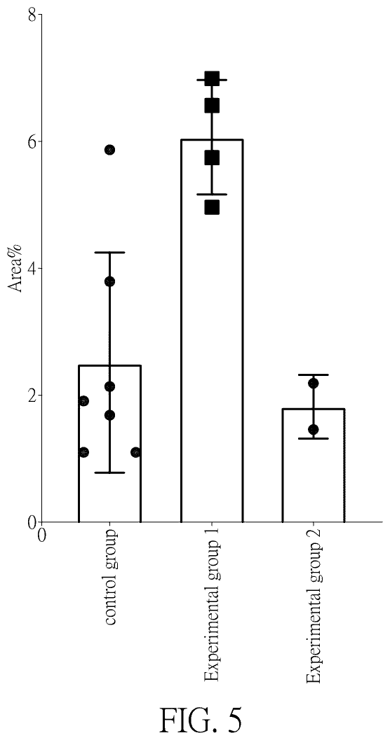

Referring to FIG. 5, FIG. 5 is a bar graph showing how intracerebral circulation efficiency is affected by a heart rate under different conditions. In the experimental process, 20 C57BL/6 mice had a minimum acclimation period of five days in a laboratory, and then were divided into three groups: the control group, Experimental group 1, and Experimental group 2. No drug was administered to the mice in the control group, the mice in Experimental group 1 were injected with isoprenaline at a concentration of 30 mg/kg/day, and the mice in Experimental group 2 were injected with propranolol at a concentration of 3.75 mg/kg/day. The drug administration continued for 1 week. Subsequently, a fluorescent tracer was injected into the cisterna magna of the mice. After 30 minutes, the mice were sacrificed, and the brain was coronally sectioned to observe an area of fluorescence diffusion within the brain.

According to the results in FIG. 5, the average heart rate of the mice in the control group was 64.8 bpm. The average heart rate of the mice in Experimental group 1 was 139.9 bpm, and the area of fluorescence diffusion was greater in Experimental group 1 as compared to the control group. In contrast, the average heart rate of the mice in Experimental group 2 was 40.5 bpm, and the area of fluorescence diffusion was smaller in Experimental group 2 as compared to the control group. Accordingly, a speed of the heart rate affects a rate at which a fluid from the subarachnoid space enters the cerebral parenchyma, thereby influencing the efficiency of cerebrospinal fluid circulation in the brain. Specifically, a faster heart rate results in a faster intracerebral circulation rate, and a slower heart rate results in a slower intracerebral circulation.

The heart rate is related to the intracerebral circulation rate. As such, when a frequency of the ultrasound stimulation matches a heart rate frequency, the frequency of the ultrasound stimulation can accelerate the intracerebral circulation rate. More specifically, when the frequency of the ultrasound stimulation is synchronized with systolic signals of the heart rate, the frequency of the ultrasound stimulation can more effectively accelerate the intracerebral circulation rate.

### Beneficial Effects of the Embodiments

In conclusion, in the circulatory system stimulation device provided by the present disclosure, by virtue of “the circulatory system stimulation device including the heart rate sensing unit, and the stimulation unit being disposed on the support unit and electrically connected to the heart rate sensing unit for providing the ultrasound stimulation,” and “the heart rate sensing unit detecting the heart rate signal, and the ultrasound stimulation being synchronized with the heart rate signal,” the effectiveness of stimulating the intracerebral circulation can be improved, and a patient is less likely to feel uncomfortable.

Furthermore, since a flow speed of an intracerebral fluid is confirmed to be related to the heart rate, the present disclosure provides the circulatory system stimulation device that combines the stimulation unit with the heart rate sensing unit. The circulatory system stimulation device of the present disclosure synchronizes the heart rate signals with the stimulation device, and delivers stimulation only when necessary (instead of continuously delivering stimulation). Specifically, when the stimulation frequency matches the heart rate frequency, the stimulation frequency can further accelerate the intracerebral circulation rate. This design enhances circulation efficiency and reduces the energy of ultrasound mechanical stimulation, thereby achieving an effect of promoting the intracerebral circulation.

Moreover, the circulatory system stimulation device of the present disclosure may be used to improve diseases that include, but are not limited to, stroke, major depression, Parkinson's disease, post-traumatic stress disorder, Alzheimer's disease, autism spectrum disorder (ASD), schizophrenia, and pain management.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A circulatory system stimulation device for stimulating a circulatory system,
comprising:
a support unit;
a stimulation unit disposed on the support unit for providing ultrasound stimulation; and
a heart rate sensing unit electrically connected to the stimulation unit;
wherein the heart rate sensing unit detects a heart rate signal, and the ultrasound stimulation is synchronized with the heart rate signal.

2. The circulatory system stimulation device according to claim 1, further comprising a control unit electrically connected to the heart rate sensing unit and the stimulation unit, so as to control a stimulation frequency of the stimulation unit based on the heart rate signal detected by the heart rate sensing unit.

3. The circulatory system stimulation device according to claim 1, wherein the heart rate sensing unit detects the heart rate signal, the heart rate signal includes a systolic signal and a diastolic signal, and the ultrasound stimulation is synchronized with the systolic signal.

4. The circulatory system stimulation device according to claim 1, wherein the support unit is an annular band.

5. The circulatory system stimulation device according to claim 1, wherein the support unit includes a cap body and a cap band, the heart rate sensing unit is disposed on the cap band, and the stimulation unit is disposed on the cap body.

6. The circulatory system stimulation device according to claim 1, wherein the stimulation unit is arranged in pairs.

7. The circulatory system stimulation device according to claim 1, wherein the circulatory system is intracerebral circulation, lymphatic circulation, or blood circulation.
